(19) United States
(12) Patent Application Publication
(10) Pub. No.: US 2025/0177293 A1
(43) Pub. Date: Jun. 5, 2025

(54) THERMAL ENERGY SYSTEM FOR ADMINISTERING ACTIVE AGENTS

(71) Applicant: ULTRAORAL LTD., RA’ANANA (IL)

(72) Inventor: DANIEL SANDERS, RA'ANANA (IL)

(73) Assignee: ULTRAORAL LTD., RA'ANANA (IL)

(21) Appl. No.: 19/044,666

(22) Filed: Feb. 4, 2025

Related U.S. Application Data
(63) Continuation-in-part of application No. PCT/IL2024/050378, filed on Apr. 17, 2024.
(60) Provisional application No. 63/461,284, filed on Apr. 23, 2023.

(51) Int. Cl. A61K 9/00; A61K 38/26; A61K 47/02; A61K 47/46

(52) U.S. Cl. CPC A61K 9/0056; A61K 38/26; A61K 47/02; A61K 47/46

(57) ABSTRACT

Described herein, according to an embodiment, is an intra-oral composition for delivery of active agents comprising: a core, the core having at least one active agent, a phase change material, an exothermic material, and optionally a permeation enhancer, optionally a bitterness blocker, sweeteners and flavorings; where the core comprising less than 0.5% water. Further described herein according to embodiments, are methods for treatment comprising administering to a subject in need thereof, an intra-oral composition for intra-oral delivery of active agents comprising: a core, the core having at least one active agent, a phase change material, and an exothermic material, the core comprising less than 0.5% water.

### CROSS REFERENCE TO RELATED APPLICATIONS

This is a continuation-in-part of International Patent Application No. PCT/IL2024/050378 filed on Apr. 17, 2024, which in turn claims benefit from U.S. Provisional Patent Application No. 63/461,284, filed Apr. 23, 2023, the contents of which are incorporated by reference herein in their entirety

### FIELD

Embodiments of the invention relate to compositions for intra-oral administration which include active agents and thermal energy systems.

### BACKGROUND

Mouthwashes/rinses and oral gels containing a broad-ranging variety of active agents/ingredients have been widely used and topically applied for many years. Many drugs or nutritional supplements have been applied topically as well inside the oral cavity in this manner. Additionally, chewing gums and intra-oral muco-adhesive patches that contain active agents on their tissue-facing surfaces have been used to deliver drugs or nutritional supplements through the intra-oral tissues.

Delivery of exogenous materials via intra-oral administration through the oral cavity, especially via the oral mucosa, is considered to be a convenient strategy in the clinic or at home with high safety, especially for patients with needle fear and swallowing difficulties. Without the degradation that occurs in the gastrointestinal tract and first-pass hepatic metabolism breakdown, intra-oral tissue delivery is a viable route of administration option for macromolecule absorption when compared to parenteral oral (PO) administration or needle-based injections.

Two systems which can be used for intra-oral administration to the oral mucosa include functional chewing gums and oral muco-adhesive patches. A chewing gum is a cohesive substance which is soft when chewed, designed to be chewed without being swallowed. A functional chewing gum includes an active agent such as a drug or nutritional supplement, which is released from the chewing gum matrix and administered to the subject while he/she is chewing the chewing gum. Chewing gum typically comprises a gum base, sweetener, softener, and flavor. An oral muco-adhesive patch is a carrier for a drug or nutritional supplement which has an adhesive for adhering to the oral mucosa, and a drug or nutritional supplement which is released when the patch is in the subject's mouth. In some muco-adhesive patches, at the point of application, the patch's adhesive secures it to the tissue, and the exogenous material contained in the patch begins to permeate through the oral tissue membranes and into the bloodstream, thereby providing a benefit to the subject.

There are various muco-adhesive polymers which have been used for the development of oral muco-adhesive or bio-adhesive patch delivery systems and include chitosan, polyacrylic acid, alginate, poly-methacrylic acid and sodium carboxymethyl cellulose. Chitosan, a cationic polymer, is widely used for its biodegradable and biocompatible properties and it undergoes electrostatic interactions with the negatively charged mucin chains thereby exhibiting muco-adhesive properties. The term bio-adhesion implies attachment of a drug carrier system to a specified biological location. The biological surface can be epithelial tissue or the mucus coating on the surface of a tissue. If adhesive attachment is to a mucus coat, the phenomenon is referred to as muco-adhesion.

A muco-adhesive patch is typically composed of four layers: an impermeable backing layer that is the basal (outer) layer and consists of water-insoluble material; an active agent reservoir, comprising an active agent such as a drug or a nutritional supplement; a semi-permeable membrane that may serve as a rate-limiting barrier; and an adhesive layer. An example of backing membrane material that is impermeable to saliva/moisture is polyethylene terephthalate/ethylene vinyl acetate (PET/EVA).

The materials used as backing layer in typical muco-adhesive patches are configured to be inert to the active agent and optional penetration enhancer present in the active agent reservoir. The impermeable backing layer on known muco-adhesive patches is designed to prevent the loss of active agent to saliva washout over extended periods of application of the patch intra-orally, to allow for diffusion of the active agent in a rate limiting and relatively slow manner through the oral tissue.

The adhesion process is complex and involves contact, consolidation and the formation of some type of bond between the polymer and the mucus. Adherence of the two materials is attained by contact between a pressure-sensitive adhesive and a surface (mucous membrane). Several polymer related factors like molecular weight, chain length, degree of cross-linking, hydration, functional groups, charge, polymer concentration and several environmental and physiological factors like contact time, mucin turnover rate and mucus viscosity affect the degree of muco-adhesion.

As mentioned above, the time to onset of pharmacological activity of the exogenous material from a typical muco-adhesive patch is very slow because of the relatively slow, passive absorption of the exogenous material through the intact skin/oral tissue membranes. For instance, in commercially available scopolamine transdermal patches, it typically takes four hours for circulating plasma levels of scopolamine to even be detected once a patch is applied, and the time to reach peak drug levels averages twenty-four hours. Carvedilol muco-adhesive patches for tachycardia typically take 8 hours to release 95% of the drug into the oral mucosal tissue.

Oral tissues are a complex series of tissues lining the oral cavity. They consist of tissue layers such as stratified squamous epithelium, basement membrane, and supporting connective tissues underneath. Besides dentition, the buccal oral tissue, in addition to the sublingual, palatal and gingival oral tissues, are part of the intra-oral tissues of the oral cavity. The buccal oral tissue consists of the outer epithelium and basement membrane. Non-keratinized stratified squamous epithelium forms the outer buccal epithelium. It is composed of mostly phospholipids as well as proteins in the form of tonofilament. The basal layer of the epithelium differentiates into replacement cells that are shed from the outermost tissue surface. The epithelium, due to its morphology and lipid structure, is considered as the major barrier for the penetration of most active agents in buccal delivery.

Apart from the presence of barrier materials between the cells of the superficial layer, the surface of the oral epithelium is normally bathed in saliva. Besides its role as a fluid in diluting and removing surface materials, saliva provides more than just a washing action and salivary mucin may contribute to enhancing the barrier layer impermeability of all the oral tissues.

There are two permeation pathways for passive diffusant/permeant transport across the oral tissues: Para-cellular and Trans-cellular routes. Permeants/diffusants can use these two routes simultaneously, but one route is usually preferred over the other depending on the physicochemical properties of the diffusant. Since the intercellular spaces and cytoplasm are hydrophilic in character, lipophilic compounds would have low solubilities in this environment. The tissue cell membrane, however, is rather lipophilic in nature and hydrophilic solutes will have difficulty permeating through the cell membrane due to a low partition coefficient. Therefore, the intercellular spaces pose as the major barrier to permeation of lipophilic compounds and the cell membrane acts as the major transport barrier for hydrophilic compounds. Since the oral epithelium is stratified, solute permeation may involve a combination of these two routes. The route that predominates, however, is generally the one that provides the least amount of hindrance to passage.

There are a number of main pathways for active permeation/diffusion of the oral tissues by the action of permeation enhancer materials. These may act by a number of mechanisms, such as increasing the fluidity of the cell membrane, extracting inter/intracellular lipids, altering cellular proteins, or altering surface mucin, or vasodilation of the blood vessels in the tissue via increasing nitric oxide levels. The greater the degree of effect, the larger the volume of diffusant permeation, the more rapid rate of diffusion and the ability of the permeation enhancer material allow for larger size molecules to diffuse through the oral tissues.

As described above, the majority of the oral tissues act as a natural barrier to rapid permeation of exogenous materials (especially those with high molecular weight), such as nutritional supplements and many drugs across the oral tissues, as transport of these exogenous materials across the oral epithelium normally occurs via passive diffusion which is a relatively slow and time-consuming process and requires a high level of patient compliance in order to be even partially effective.

It is difficult for the user to keep liquid/gel agents, such as nutritional supplements or drugs inside the mouth for lengthy periods of time. Lozenges, chewing gum carriers and mucoadhesive delivery systems (patches) have been formulated to try to overcome this treatment exposure time limitation. This difficulty, and the inherent difficulty to transport active agents through oral tissues, are both reasons that intra-oral delivery of active agents is not a widely utilized mode of administration.

### SUMMARY

Described herein, according to an embodiment, is an intra-oral composition are compositions for intra-oral delivery of active agents comprising: a core, the core having at least one active agent, a phase change material, and an exothermic material, the core comprising less than 0.5% water.

Further described herein according to embodiments, are methods for treatment comprising administering to a subject in need thereof, an intra-oral composition for intra-oral delivery of active agents comprising: a core, the core having at least one active agent, a phase change material, and an exothermic material, the core comprising less than 0.5% water.

Further described herein (though not limited those described) according to some embodiments are various nutritional supplements or active pharmaceutical ingredients APIs (drugs) that may be incorporated for intra-oral applications via either a chewing gum or muco-adhesive patch of the present invention.

### BRIEF DESCRIPTION OF THE FIGURES

FIG. 1 is a flow diagram depicting a method for administration of an active agent using a composition comprising a thermal energy system (TES) according to an embodiment;

FIG. 2 is a graph showing the melting point of mixtures of light paraffin oil with beeswax at various concentrations by weight;

FIGS. 3A and 3B are graphs showing temperature change of mixtures of exothermic anhydrous salts with water over time, in mixtures without phase change material (PCM) in FIG. 3A and with PCMs in FIG. 3B.

### DETAILED DESCRIPTION

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides or peptides or proteins or portions or fractions thereof are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Described herein, according to an embodiment, are compositions for intra-oral delivery of active agents comprising: a core, the core having an active agent, a phase change material, an exothermic material, and a permeation enhancer, preferably wherein the exothermic material is in the form of an anhydrous salt and the core comprising less than 0.5% water. Optionally, the composition is in the form of a chewing gum, and further comprises a chewing gum base. Optionally, the composition is in the form of a mucoadhesive patch.

According to an embodiment, such compositions for intra-oral administration comprise heat-generating excipients, configured to generate heat and increase temperature in the oral cavity over the duration of delivery. It is suggested that increased temperature at the site of administration will increase permeability of oral tissues, thereby allowing for higher absorption of active agents. Optionally, the composition is in the form of a chewing gum or muco-adhesive patch.

In a previous study (AAPS PharmSciTech, Vol. 12, No. 2, June 2011) it was shown that heating various active agents which were used as diffusants (buspirone, bupivacaine, antipyrine and caffeine) was demonstrated across porcine oral tissues at five different temperatures of 23° C., 30° C., 37° C., 45° C., and 52° C. where permeation across the oral buccal mucosa follows a relationship similar to the Arrhenius equation (first order kinetics) where:

$${D}_{T} = {D}_{0} \times {e}^{\frac{- {E}_{A}}{RT}}$$

DT the diffusion coefficient at a certain temperature (cm²/sec)
D0 the theoretical maximum diffusion coefficient at infinite temperature (° K) (arbitrary value, preexponential factor)
EA activation energy of diffusion (J/mol)
R the universal gas constant (83144 J/mol° K)
T the temperature of interest (° K)

Permeation of all diffusants tested was found to increase by a factor from 1.4 times up to 2.4 times for each incremental rise in experimental temperature of approximately 7° C. An exponential relationship was therefore observed between the temperature and the permeability of each of the tested four diffusants across the oral tissue barrier. Irreversible effects, in the oral tissues, only occurred above 68° C. Hence, the temperature range that was tested (23°−52° C.) can be considered appropriate and safe for oral tissues.

Without being bound by theory, it is suggested that heating the oral cavity by administering compositions disclosed herein will increase temperature at an oral tissue thereby allowing for enhanced permeation because of the increased energy of the penetrant crossing into the oral tissue. Furthermore, vasodilation of the subcutaneous blood vessels as a homeostatic response to a rise in oral tissue temperature also plays an important role in enhancing the diffusion and delivery of active agents to the oral tissue. Other advantages of heating the oral cavity may include: a. release of a greater volume of the active agents from the composition; b. heating of the saliva increasing the solubility of the agent in the saliva, and a reduction in saliva viscosity, increasing saliva flowability; c. increases the concentration of the active agents in the saliva; d. increases overall intra-oral tissue exposure to the formula ingredients; e. increases tissue membrane fluidity; f. increases permeation/diffusion of the ingredients into and through the tissue membranes; g. increases the flow of ingredients from the tissues into the blood stream; h. increases bioavailability of the active agents to the body's organs.

Described herein are compositions for intra-oral delivery of active agents, configured to enhance penetration of the oral tissues by generating heat through a thermal energy system (TES). A TES comprises an exothermic material (EM) and a phase change material (PCM). An EM is a material which releases heat upon contact with water/moisture. A PCM is a material which changes phases from solid to liquid in a predetermined temperature range, preferably between 40° C. to 48° C., preferably 44° C. to 48° C.

When the EM is an anhydrous salt, upon administration of the composition, a hydration reaction occurs, and heat is generated inside the composition and transferred to the oral tissue during the duration of intra-oral application. The compositions are configured to generate heat at specific temperature ranges through the sequential release of exothermic energy upon exposure of an exothermic material (EM) such as an anhydrous salt to saliva present in the mouth of a subject. The heat generated by the reaction of the anhydrous salt with saliva provides heat to oral tissue in the oral cavity, and also causes the PCM incorporated within the composition to absorb the thermal energy released by the adjacent activated EM and change phase (it is formulated to phase change within the temperature range generated by the EM), thereby storing thermal energy. The PCM then gradually releases heat as it cools, by phase change, to provide heat to the oral tissues. This process of phase change may occur continuously over parts of the composition during administration of the composition, so as to prolong the heating effect of the TES. This process repeats/cycles over the duration of oral application acting as a “cascading” thermal energy system as the EM is gradually hydrated, it sequentially releases heat in a relatively controlled manner (based on its molar concentration in the formulation). The presence of PCM in the composition retards the cooling that occurs to a greater extent in an equivalent composition which does not contain a PCM. This prolonged time in which the TES provides heat to the composition and to the oral cavity, provides extended and sustained increased diffusion of the active agent for the duration of the application.

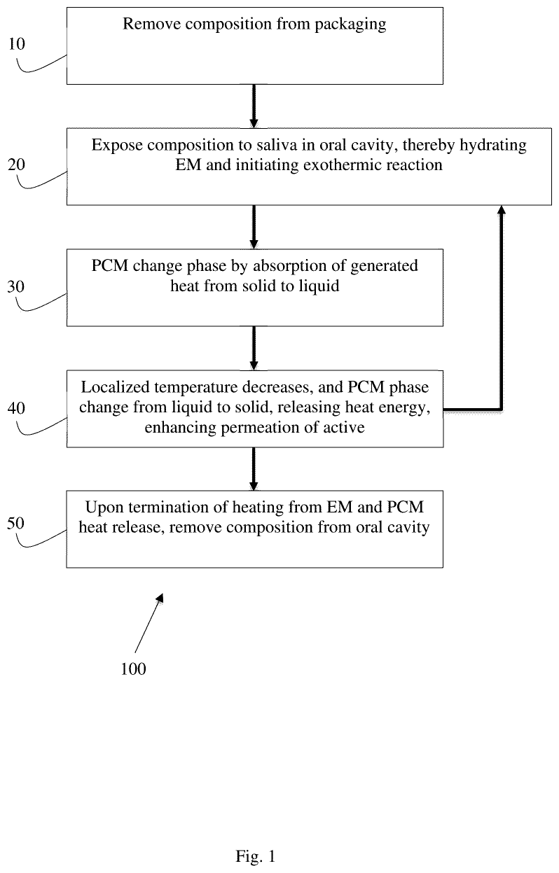

In addition, some embodiments relate to methods for administration of active agents comprising administration of compositions comprising an active agent and a TES to an oral cavity of a subject in need thereof. An embodiment of such method is described in method 100, which is graphically depicted in FIG. 1. Method 100 comprises block 10, which comprises removing the composition from a packaging. Preferably, the packaging is an airtight packaging. A packaging comprising a blister pack with high density polyethylene backing (such as a Tyvek® heat sealed blister pack with individual blister cavities for each piece of gum) which does not allow the passage of moisture and/or air from the environment outside of the packaging into the space encompassing the composition within the packaging. Additionally, a pre-coating that entirely seals the core portion of the gum and that is impervious to water, and optionally as a second final hard coating may be applied to the gum cores prior to packaging to prevent the passage of moisture and/or air the environment even when the gum is removed from either the blister pack packaging or alternatively when placed in a sealed bottle packaging.

Method 100 further comprises block 20, comprising exposing the composition to saliva in the oral cavity. When the composition is a chewing gum, exposing the composition to saliva is performed by introducing the composition into the mouth of a subject, and beginning to chew the chewing gum. When the composition is a mucoadhesive patch, exposing the composition to saliva ingress is performed by adhering the mucoadhesive patch to a mucosal membrane of the oral cavity, wherein the non-tissue facing side of the patch is a semi-permeable membrane structure. While contacting saliva, EM within the composition will hydrate, thereby generating heat through an exothermic reaction. The Adjacent PCM incorporated within the composition absorbs the thermal energy released by the adjacent activated EM and change phase (it is formulated to phase change within the temperature range generated by the EM), thereby storing thermal energy. The PCM then gradually releases heat as it cools, by phase change, to provide heat to the oral tissues. This process of phase change may occur continuously over parts of the composition during administration of the composition, so as to prolong the heating effect of the TES of the muco-adhesive patch. This prolonged time in which the TES provides heat to the composition and to the oral cavity, provides extended and sustained increased diffusion of the active agent for the duration of the application.

Method 100 further comprises block 30, comprising phase change of PCM by absorption of heat from the composition, from a solid to a liquid.

Method 100 further comprises block 40, comprising phase change of PCM from liquid to solid, releasing heat energy in the composition and oral tissue. This phase change occurs when localized temperature decreases below a certain threshold limit. Releasing heat in this step prolongs the duration of heat available in the oral cavity as a result of the composition.

After block 40, more of the EM within the composition may be exposed to saliva, thereby hydrating the EM and releasing heat via an exothermic reaction in accordance with block 20, which in turn may lead to continued phase change in accordance with blocks 30 and 40. When the composition is a chewing gum, more of the EM within the composition may be exposed to saliva by continued chewing of the composition by the subject. When the composition is a muco-adhesive patch, more of the EM within the composition may be exposed to saliva by the continued penetration of the saliva into the muco-adhesive patch, as will be described below.

Method 100 further comprises block 50, comprising termination of heat release from the EM and PCM, thereby the composition reverts equilibrium at the temperature of the oral cavity. At this point, the composition may be removed from the oral cavity. In the case of a chewing gum, the composition may be removed from the mouth and disposed of. In the case of a mucoadhesive patch, the composition may be removed from the mouth entirely.

Compositions, according to embodiments of the invention, comprise an active agent, or multiple active agents. Active agents may optionally be microencapsulated, as addressed below with regard to PCM. Active agents may optionally be comprised within a food grade macro-porous or micro-porous granule/particle as mentioned with regard to PCM.

As mentioned, PCMs and EMs together allow for prolonged heat application to oral tissues while the disclosed compositions are being administered. Many materials may act as PCMs, but preferred PCMs were found to absorb and release thermal energy during the process of melting and re-solidifying at a temperature range that is relevant to the heat released by the EMs. These PCMs have latent heat storage capacity.

According to an embodiment, PCMs used in compositions described herein undergo phase change at a temperature of 40° C. to 48° C., preferably 44° C. to 48° C.

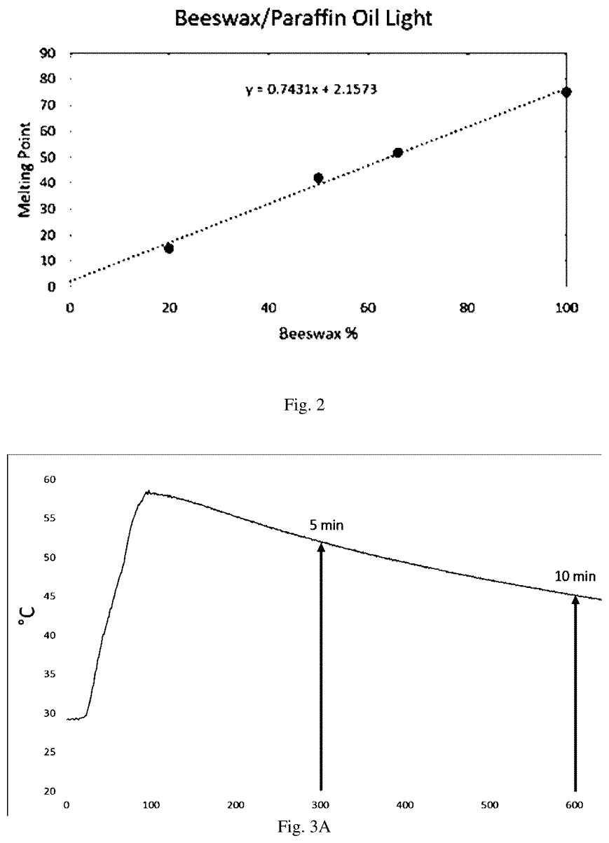

According to an embodiment, the PCM is selected from a group consisting of beeswax, glycerol, polyethylene glycol, and paraffin oil. Optionally, beeswax, in combination of an agent which lowers its melting temperature, is used as a PCM, optionally paraffin oil, glycerol, or polyethylene glycol. Optionally, a combination of beeswax and paraffin oil is used as a PCM. Optionally, the beeswax and paraffin oil are used in a ratio of between 1:1 and 2:3. FIG. 2 shows a graph showing melting point of mixtures of beeswax and light paraffin oil at various ratios. As can be seen, mixtures of beeswax and paraffin oil having about 40% to about 80% beeswax have melting points between about 32° C. and 62° C., which may be potentially relevant for compositions for intra-oral delivery.

According to an embodiment, the PCM is present in the core in an amount of 1% to 10% by weight. Optionally, the ratio by weight of PCM to EM is about 1:10.

According to an embodiment, the PCM is encapsulated using “microencapsulation.” Microencapsulation is an advanced food processing technology where any compound can be encapsulated inside a particular material, making a tiny sphere of diameter ranging from one micron to several hundred microns. The compound or material which is encapsulated is encapsulated by another substance known as an encapsulant, wall or shell material. Encapsulants can be either polymeric or nonpolymeric materials like cellulose, ethylene glycol, and gelatin. There are several techniques used for microencapsulation. Fluidized bed coating, spray cooling, spray drying, extrusion, and coacervation some microencapsulation processes.

On the basis of the physical and the chemical properties of the interior being encapsulated, composition of the shell material and the microencapsulation method used, various types of capsules are obtained: simple sphere surrounded by the wall material, capsules with irregular interior, multiple distinct interiors within a continuous coating of wall material, multiwalled microcapsules and interior particles embedded within the matrix of wall material. Depending on the kind of coating material used, different techniques are used to produce the microcapsules and these techniques lead to differences in the properties of the capsules.

The selection of a particular technique depends upon the properties of the interior material, encapsulant, and different properties and morphology of the capsules desired. The characterization and optimization of efficient and successful encapsulation can be done by studying the encapsulation efficiency and various properties of the capsules like morphology, size, hydrophobicity, hygroscopicity, solubility, surface tension, thermal behavior, hydrophobicity, thermal and mechanical properties.

Microcapsulation coating materials may include silicone dioxide, chitosan, carbohydrates such as starch, sucrose, maltodextrin, modified starch, cyclodextrin; lipids such as beeswax, diacylglycerols; gums such as gum acacia, gum arabic, agar, guar, carrageenan; and proteins such as gluten and casein. The coating material or the wall/shell material used in micro-encapsulation should be such that it is able to form a cohesive film on the interior, stabilize it, and provide strength to the capsules, inert, so that it has no reaction with the interior material, does not provide any specific taste to the product, impermeable and with ability to release the interior at a specific time and place, upon specific treatment.

Spray drying is a technique in which a feed solution, which is a mixture of the interior material and the wall/shell material, is atomized and formed into a mist inside a chamber, where hot air is applied to convert the mist into powder, a technique where droplets of solution/suspension are converted into a dry powder by the evaporation of the solvent/liquid. Depending on various factors like the characteristics of the feed solution and operating conditions, powder of varied particle size can be produced. In spray drying, the interior material, that is, the material of interest, becomes trapped in the dried powder.

Spray cooling method is another micro-encapsulation technique. Spray cooling is very similar to spray drying in operation, the major difference being the use of cold air in it. Here, a mixture of interior material and wall material is atomized to form a mist inside a chamber, inside which cold air flows. The low temperature within the chamber results in solidification of the micro droplets, leading to the formation of microencapsulated powder.

Coacervation is another micro-encapsulation technique. Coacervation is a simple technique which involves formation of a homogeneous layer of the polymeric wall material around the interior material. This is achieved by altering the physicochemical properties of the wall material by change in temperature, pH, or ionic strength. Here, the interior material and the wall material are mixed to form an immiscible solution. Then, phase separation is carried out by changing the ionic strength, pH, or temperature to form coacervates, which are tiny liquid droplets, consisting of polymer-rich dense phase. These coacervates then surround the core material, forming the microcapsules. Electrostatic interaction between two aqueous media is responsible for liquid to gel transition, that is, ionic gelation, hence, leading to the formation of coacervates. This technique is basically used for encapsulating hydrophilic molecules. Several studies have been reported showing successful use of this technique in micro-encapsulation.

Extrusion technology for microencapsulation can be used for producing highly dense microcapsules. To use this method, the interior and the wall material should be immiscible. Here, the core and the wall materials are passed in such a way that the wall material surrounds the core, and they are passed through concentric nozzles, thus forming droplets containing the core surrounded by the wall material. Then solidification is done either by cooling or using an appropriate gelling bath wherein the droplets fall and solidify due to formation of complex. The encapsulates formed using this method are relatively larger in size than formed using any other method and also, this technology is useful with limited wall materials.

Encapsulation using emulsification technique is done by dispersing the interior in an organic solvent, containing the wall material. The dispersion is then emulsified in the oil or water, to which emulsion stabilizer is added. Encapsulation of the interior occurs by formation of a compact polymer layer around it, by evaporation of the organic solvent. This is one of the frequently used techniques of encapsulation as the procedures involved are simple.

Microencapsulation of PCM may provide (a) extended shelf life of any or all of the various formulas ingredients, (b) improved thermal stability of the PCM, (c) more uniform distribution/dispersal of the PCM and therefore more uniform heating activation of the composition, (d) improved controlled release and extended release time of the PCM when chewing these formulas inside the oral cavity or releasing them from an impregnated intra-oral muco-adhesive patch (e) improved resistance to release of the core materials as required to maintain the core materials inside the capsule shells even upon exposure of the composition to stimuli like heat or pressure or hydration. Said coated material micro-capsules may range in diameter size from 1 to 200 microns or even 1-50 microns in size.

According to an embodiment, a PCM may be comprised within a food grade macro-porous or micro-porous granule/particle to provide: (a) extended shelf life of the PCM, (b) improved thermal stability of the PCM, (c) more uniform distribution/dispersal and therefore more uniform heating activation the composition, (d) readily release with better controlled release and longer release time of the PCM inside the oral cavity, (e) improved release of the PCM as required. Said substantially spherical-shaped granules/particles may range in diameter size from 50 to 200 microns or even 1-50 microns in size. Such particles may comprise maltodextrin and/or food grade polystyrene.

Preferred EM include pharmaceutically acceptable salts in anhydrous form, which when come in contact with water, release heat. The EM used in a composition may be, but are not limited to, anhydrous salts (for example, where all the water molecules in the salt have been stripped away by a meticulous drying process) such of magnesium citrate, magnesium sulfate, strontium bromide (SrBr₂) strontium chloride (SrCl₂), trisodium phosphate (Na₃PO₄), magnesium chloride (MgCl₂), calcium sulfate (CaSO₄), and zinc sulfate (ZnSO₄). A composition may comprise one EM or multiple EMs, in combination in various ratios and amounts.

Magnesium citrate and magnesium sulfate are preferred anhydrous salts. When these salts are preferably in a fully anhydrous form, upon exposure to moisture, they undergo a relatively rapid hydration reaction in a matter of seconds that can yield a relatively high energy output (from 45-90 kilojoules or more per mole). The amount of heat produced in such a reaction when a composition in contacted with water will depend on several factors including: a. the EM ease of dissociation and subsequent association with water molecules to thereby produce a relatively robust exothermic chemical reaction b. the degree to which they are maintained fully anhydrous in storage prior to manufacture, during manufacture and post-production within the compositions. As EM salts including magnesium citrate and magnesium sulfate are hygroscopic, care should be taken to isolate EM from water throughout the manufacture and storage of compositions. In order to prevent absorption of water from air by hygroscopic EM, one effective method is by coating composition cores with coatings which prevent moisture and/or air from penetrating the coating, and by packaging compositions in packaging which prevents or limits exposure of air to the compositions. By limiting contact of intra-oral compositions to air and moisture before administration, a maximal amount of heat may be generated upon administration to the oral cavity.

According to an embodiment, a core of a composition comprises less than 0.5% water.

According to an embodiment, the EM is encapsulated using microencapsulation. Microencapsulation may be performed according to the methods described above. EM may be encapsulated by a barrier which limits contact of saliva with the EM. In case of a chewing gum, for example, as the composition is chewed, physical agitation of the chewing gum degrades the encapsulation, thereby exposing EM to saliva. An advantage of encapsulation of EM in chewing gum is that as the chewing gum is being chewed by the subject, the amount of time in which EM contacts water and generates heat is prolonged relative to comparable compositions in which the EM is provided in non-encapsulated form. EM optionally may be comprised within a food grade macro-porous or micro-porous granule/particle as mentioned with regard to PCM.

According to an embodiment, the composition comprises a core and one or more coatings. Preferably, the coating contains a barrier which prevents air and/or water vapor from penetration from the environment outside of the barrier, to the core of the composition within the barrier.

The coating may be comprised of two layers where the inner layer is a pre-coat layer, which is in direct contact with the core, and a final coat layer, which coats the pre-coat layer. Optionally, the pre-coat layer completely coats the core and provides a waterproof seal. Optionally, the pre-coat layer comprises either coating the center core with sugar free shellac and then precoating for another coating with a sugar free gumming agent that smooths the center core surface and allows for homogeneous coating results, even for cores with uneven surfaces. Alternatively, coat with shellac, or surface treatment with a sugar free liquid glazing and sealing agent and pre-coating with sugar free shellac for a further coating.

The final coat layer may comprise flavorings, sugarless sweeteners and bitterness blockers applied using a water spraying solution that is air dried utilizing a dragee panning application or other coating method. The weight of both pre-coat and final coat layer together comprise approximately 10%-50% of the weight of the core.

The compositions described herein, in addition to comprising a TES, also may comprise permeation enhancer materials (PEs) in their cores to further improved permeation/diffusion of the active agent contained in the composition into and through the intra-oral tissue membranes. A PE is a chemical which facilitates penetration into or through the poorly permeable biological membranes. This enables throughout the application a further increased rate of permeation/diffusion and increased total volume of permeation/diffusion of the active agents into and through the oral tissues and then into the circulatory system of the body as well, thereby providing improved/superior local and/or systemic bioavailability of the active ingredients/agents to the tissues of the body as well as enhanced clinical outcomes and health benefits.

According to an embodiment, the permeation enhancer is selected from the group consisting of: mannitol, menthol, and an essential oil. Optionally, the essential oil is selected from the group consisting of: peppermint oil, sage oil, and eucalyptus oil.

An example of a class of permeation enhancer materials that may be incorporated in the compositions of the present invention and that may be coated with various food grade micro-encapsulated coatings, are essential oils (EO). EOs are oily, aromatic liquids extracted from aromatic plant materials, and are natural products which consist of complex blends of many aromatic-smelling volatile compounds. The predominant compounds within these blends are terpenes, terpenoids, phenylpropanoids, as well as minor amounts of miscellaneous volatile organic compounds. The terpene family is predominant, and phenylpropanoids, when they appear, are responsible for the characteristic odor and taste released on heating and/or light activation of the formulas inside the oral cavity.

As permeation enhancers, EOs can increase the delivery of small drug compounds into the skin by interacting with the intercellular lipids of the tissue through physical processes including extraction, fluidization, increased disorder, and phase separation. While EO's and their constituents can penetrate through the skin or oral tissue into the blood stream, in general they affect the membranes temporarily and are also easily excreted from the body within the urine and feces.

Due to the fact that permeation/diffusion/penetration enhancer materials are important to support percutaneous and intra-oral absorption of drugs, nutritional supplements or other oral treatment agents by lipid disruption, protein modification or partitioning promotion functions reducing the barrier function of the skin, they allow molecules to pass through the layer of the tissues faster. The most key point for safe and effective delivery through the oral tissues is the selection and use of permeation/penetration enhancer materials that cause a relatively temporary and reversible reduction in the barrier function of the oral tissues. As a class, essential oils have a permeation enhancing activity profile.

One example in this class of permeation/diffusion enhancer materials that may be incorporated into the various compositions of the present invention is menthol, the main component of peppermint oil. Menthol (also “mint camphor”), is a volatile oil extract derived from the genus Mentha (mint), is widely available in natural and synthetic forms.

Menthol has been demonstrated to has been shown to increase blood flow to the area where it is applied. A study in Microvascular Research found that a 4 percent menthol solution caused blood vessels to widen, which increases blood flow thereby effectively increasing the permeation/diffusion of various active ingredients across the oral tissue barrier.

Another example of a permeation enhancer is Eucalyptus Oil. It contains 1,8-cineole, a monoterpene cyclic ether which can enhance penetration/permeation of both lipophilic and hydrophilic compounds. Eucalyptus Oil which contains polyphenols was found to be highly effective, causing a near 30-fold increase in the drug permeability coefficient. Salvia Officinalis (Sage Oil) has also been shown to be high in polyphenols, an antioxidant, antibacterial, anti-inflammatory and permeation enhancer.

According to an embodiment, a PE is included in the core of a composition, at an amount of between 1% and 5% by weight of the core.

According to an embodiment, the composition for intra-oral delivery of active agents is in the form of a chewing gum which may be a synthetic or naturally derived chicle gum base. Synthetic versions of the gum base are composed of polymers, plasticizers and resins. The polymers may be for example polyvinyl acetate, butadiene-styrene, or polyisobutylene. The chewing gum base is an inert, non-water-soluble substance which provides the chewing gum its consistency, allowing it to be chewed, while maintaining sufficient elasticity to allow the chewing gum to maintain cohesion as one piece while chewing in the mouth of a subject. Chewing gum base preferably comprises a resin, a polymer/elastomer, and a plasticizer. Resin are responsible for the chewiness of the gum and are typically hydrophobic. Elastomers are polymers which add flexibility and plasticizers improve chewing softness and elasticity.

According to an embodiment, the composition is formed using a gum base having low salt content, preferably 5%+/−2 by weight. Preferably, the composition has a salt content of no more than 25%.

A bitterness blocker is a compound which interacts with the molecular pathway of bitterness. According to an embodiment, a bitterness blocker is added to compositions to reduce bitterness of the potentially bitter-tasting EM. Optionally, the bitterness blocker is selected from the group consisting of Neohesperidine, Brazzein, Black Pepperdine, Cayenne Pepper, Thaumatin, Dipotassium Glycyrrhizinate.

Flavoring may be incorporated in the composition. Exemplary flavorings include, but are not limited to: vanilla mint, Turkish coffee, cappuccino, macchiato, mocha, menthol, mint, strawberry, strawberry mint, blueberry, apple, apricot, banana, butterscotch, caramel, caramel vanilla, cherry, cinnamon, grape, peach, pineapple, honey, melon, lemon. Preferably, the flavoring is free of water.

According to an embodiment, the composition for intra-oral delivery of active agents is in the form of a muco-adhesive patch. The muco-adhesive patch according to an embodiment, comprises preferably three parts: a muco-adhesive layer, which binds the patch to oral tissue; a drug reservoir, comprising a core as described herein, comprising an active agent and a TES, and a semi-permeable membrane which allows flow of water/saliva from the oral cavity to contact the drug reservoir. Upon application, the muco-adhesive layer is affixed to the oral tissue, and the semi-permeable membrane faces the oral cavity, with the reservoir being located between the muco-adhesive layer and the semi-permeable membrane.

A muco-adhesive patch comprises a muco-adhesive layer which binds the patch to oral tissue, preferably the muco-buccal tissue (inside of cheek). There are various muco-adhesive polymers which may be used for the muco-adhesive layer including, but not limited to: chitosan, polyacrylic acid, alginate, poly-methacrylic acid and sodium carboxymethyl cellulose. Chitosan, a cationic polymer, is widely used for its biodegradable and biocompatible properties and it undergoes electrostatic interactions with the negatively charged mucin chains thereby exhibiting muco-adhesive properties. At the point of application, the patch's muco-adhesive layer secures it to the tissue, and the exogenous material contained in the patch will begin to permeate through the oral tissue membranes and into the bloodstream.

The adhesion process is complex and involves contact, consolidation and the formation of some type of bond between the muco-adhesive layer and the mucus. Muco-adhesion or bio-adhesion is defined as “the state in which two materials are adhered together, which implies attachment of a drug carrier system to a specific biological location”. Adherence of the two materials is attained by contact between a pressure-sensitive adhesive and a surface (mucus membrane). Several polymer related factors like molecular weight, chain length, degree of cross-linking, hydration, functional groups, charge, polymer concentration and several environmental and physiological factors like contact time, mucin turnover rate and mucus viscosity affect the degree of muco-adhesion. as saliva can seep through the permeable or semi-permeable outer side backing layer into the inner layers of the patch containing the various formula materials embedded on the tissue-facing side of the patch.

The patch's semi-permeable membrane layer structural design can be modified to provide a spectrum/range of either permeability or semi-permeability so as to allow for varying rates of moisture/hydration exposure (slow or rapid saliva/moisture introduction) from the outer surface of the patch (in contact with the saliva) to the inner side layers of the patch containing the core and therefore varying rates of moisture activation of the EMs (optionally encapsulated) that are embedded in the inner interior of the patch. The semi-permeable membrane made be made of polyethylene, polypropylene, polycaprolactone, ethyl cellulose, and plasticizers such as glycerin, propylene glycol, polyethylene glycol, triacetin or other plasticizers.

According to an embodiment, disclosed is a method for treatment of a disease or alternatively, as a prophylactic/preventative to maintain optimal health, comprising administering to a subject in need thereof, a composition for intra-oral administration as described herein. Various active agents which may be used in intra-oral compositions, and diseases which may be treated using compositions for intra-oral administration are described below. Optionally, the active agent is a drug or a nutritional supplement. A drug is a medicine which claims to have a physiological effect when introduced to a human body or living organism, used to prevent, diagnose, treat, or relieve symptoms of a disease or abnormal condition. A nutritional supplement is a product introduced to a human body or living organism comprising a dietary ingredient, which may include a vitamin, a mineral, an herb, an amino acid, enzymes, pre or post or probiotics or other nutritional substances to improve nutrition and overall health.

According to an embodiment, the intra-oral composition comprises an active agent that may be selected from those listed in Table 1. According to an embodiment, the intra-oral composition can be used in a method for treatment to treat an indication as listed in Table 1.

TABLE 1

| Active agent | Indication |
|---|---|
| Propolis (or propolis extract), Caffeic acid | Periodontal disease (periodontitis, peri- |
| phenethyl ester (CAPE), Neohesperidin | implantitis), recurrent aphthous ulcers, oral |
| dihydrochalcone, Coenzyme Q10 with | candidiasis, caries, halitosis, cardiovascular |
| bioperidine, stevia, xylitol, curcumin, | disease, respiratory disease, Diabetes |
| spirulina, coconut oil, brazzein, thaumatin, | mellitus, Alzheimer's Disease, Fronto- |
| L-arginine, L-citrulline, mannitol, PQQ, | Temporal dementia, dementia, Parkinson's |
| niacinamide, , menthol, anhydrous | Disease, microbial disease, biofilm |
| magnesium sulfate, anhydrous magnesium | formation, inflammation, |
| citrate. | |
| Propolis (or propolis extract), L-carnosine, | Neuroinflammation, cognitive decline, brain |
| liposomal apigenin, astaxanthin, magnesium | infarction, cerebral ischemia, brain edema, |
| L-Threonate, Coenzyme Q10 with | sciatic nerve lesions, neuropathic pain, |
| bioperidine, quercetin, benfotiamine, | cognitive decline, mood disorders, improved |
| inosine, mannitol, PQQ, rosmarinic acid, | mental performance/mental acuity, memory |
| ginkgo biloba, huperzine A, Neohesperidin | recall. |
| dihydrochalcone, melatonin, pomegranate | |
| seed oil, anthocyanin, alpha GPC, bacopa | |
| monnieri, Gotu kola, marigold, curcumin, | |
| aloe vera, menthol, L-tryptophan, L-leucine, | |
| L-lysine, anhydrous magnesium sulfate, | |
| anhydrous magnesium citrate. | |
| Propolis (or propolis extract), | Obesity, stress, insulin resistance/type II |
| Neohesperidin dihydrochalcone, naringin, | diabetes, cardiovascular disease, chronic |
| green tea extract, chitosan, ephedra, green | kidney disease, rheumatoid arthritis, |
| coffee extract, inosine, liposomal apigenin, | metabolic syndrome, gut microbiome |
| benfotiamine, berberine, vitamin D, aloe | dysbiosis, Crohn's disease |
| vera, ginger, sage oil, fenugreek, 7-keto- | |
| DHEA, folic acid, vitamin B12, milk thistle, | |
| black seed oil, L-carnitine, conjugated | |
| linoleic acid, ashwagandha, brazzein, | |
| thaumatin, niacinamide, alpha lipoic acid, | |
| vitamin C, zinc, curcumin, coconut oil, | |
| menthol, anhydrous magnesium sulfate, | |
| anhydrous magnesium citrate. | |
| L-theanine, liposomal apigenin, chrysin, | Peak mental acuity/mental performance |
| magnesium L-Threonate, Vitamin B12, | |
| Coenzyme Q10 with bioperidine, | |
| Neohesperidin dihydrochalcone, brazzein, | |
| thaumatin, coconut oil, anhydrous | |
| magnesium sulfate, and anhydrous | |
| magnesium citrate. | |
| One of Semaglutide/Exenatide/Liraglutide, | Weight loss, Diabetes type II, Metabolic |
| Dulaglutide with Neohesperidin | syndrome |
| dihydrochalcone; black seed oil, brazzein, | |
| thaumatin, Coenzyme Q10 with bioperidine, | |
| mannitol, coconut oil; anhydrous | |
| magnesium sulfate, anhydrous magnesium | |
| citrate. | |

Other APIs (drugs) that when given orally are well known to have poor absorption into the organs and tissues of the body after passing through the digestive tract and the metabolism in the liver. These may include classes of drugs including glucagon like peptide-1 (GLP-1) receptor agonists, beta blockers, calcium channel blockers, ACE inhibitors, opioids, NMDA receptor antagonists, hormones, phosphodiesterase 5 (PDE 5) inhibitors, opioid antagonists, H1 receptor antagonists, and benzodiazepines.

GLP-1 receptor agonist may be selected form the group consisting of: semaglutide, dulaglutide, exenatide, liraglutide, tirzepatide, and lixisenatide.

Opioids may be selected from the group consisting of: morphine, loperamide and oxytocin. MDA receptor antagonists may include ketamine. Hormones may include progesterone and testosterone. PDE 5 inhibitors may be selected from the group consisting of: sildenafil and tadalafil. Opioid antagonists may include naltrexone. H1 receptor antagonists may include promethazine. Opioid receptor agonists may include loperamide. Benzodiazepines may include triazolam.

Of particular interest are the GLP-1 class of drugs. For example, semaglutide is normally injected subcutaneously, where the subcutaneous bioavailability of semaglutide is approximately 89%, the highest among GLP-1 receptor agonists in humans. Many patients find this delivery method objectionable and there can be complications with subcutaneous infections at the needle injection site. When taken parenteral orally, bioavailability is considerably lower, i.e., 0.8%.

The mean total surface area of the adult mouth is 214.7±12.9 cm² whilst the surface area of the sublingual region of the floor of the mouth is around 20-25 cm².

The sublingual region due to the proximity of the tongue is an uncomfortable area to apply materials. Sublingual delivery of GLP-1 drugs (e.g. semaglutide) involves the application of a gel that is inserted under the tongue. Typical residence time of the applied gel on the sublingual mucosal tissue is around one minute and due to the very muscular and active tongue, it is readily displaced and swallowed.

The current invention lends itself well to the delivery of GLP-1 drugs (e.g. semaglutide//exenatide/liraglutide, dulaglutide) via thermodynamically activated functional chewing gums or muco-adhesive patches. Compared to the sublingual delivery method, the present invention provides better and more comfortable acceptability by the user, as well providing a much larger available mucosal tissue surface area for the absorption of these drugs through the oral tissue membranes and into the robust circulatory system in the oral cavity.

The oral cavity provides a habitat for approximately seven hundred microbial species forming complex and dynamic multispecies biofilms, also referred to as ‘dental plaque’.

The oral gram-negative anaerobic bacteria Porphyromonas gingivalis is typically a late colonizer of subgingival biofilms and has been correlated with several destructive periodontal diseases, including periodontitis and peri-implantitis.

In addition, dental biofilm and specifically P. gingivalis have been found to migrate (translocate) from the oral cavity and seed other organs in the body (especially in patients with active chronic periodontitis) and have been associated with significant systemic diseases such as: cardiovascular and respiratory disease, diabetes mellitus, Alzheimer's/dementia (P. gingivalis increased the permeability of the Blood Brain Barrier and crosses the BBB), and recently, P. gingivalis found in the lungs, constituting a high-risk for developing severe illness due to Severe Acute Respiratory Syndrome Coronavirus 2 (SARS-COV-2) infection and associated with higher rates of morbidity.

To increase its clinically efficacy across a broad range of uses (pre and post dental procedures, prophylactically and in the face of chronic pathogenic disease like periodontitis and peri-implantitis) the functional chewing gums or intra-oral muco-adhesive patch applications to treat the oral cavity itself should ideally selectively target the specific pathogenic micro-organisms associated with said chronic disease conditions (without disturbing the healthy commensal micro-organisms in the mouth) and for both pre and post dental procedures be both palliative (soothing) and anti-inflammatory to the oral tissues.

Clinical value can be provided by use of compositions for intra-oral delivery of active agents described here, incorporating active agents in the core of functional chewing gum and oral muco-adhesive patches to treat the oral cavity that are safe and provide biofilm inhibition so that on application, said composition could provide lasting reduction of intra-oral levels of the pathogen P. gingivalis and other similar oral pathogens (of the “Orange and Red complex” and others) for a period of several days or more post-application.

The bacterial members that comprise the early colonizers of the gingival crevice are those with the capacity to adhere to the pellicle of the tooth and are considered only moderately pathogenic and primarily Gram-positive. The resulting foundation of early biofilm allows access to a bridging community of bacteria known as the “Orange complex” which have been found to be capable of causing periodontal pathology (e.g., F. nucleatum). Once the “Orange complex” or bacteria is established, the “Red complex” of bacteria colonize the plaque/biofilm. The “Red complex” bacteria are primarily Gram-negative, contain endotoxin (gingipains), and are well described for their highly pathogenic features (e.g., P. gingivalis).

In particular, these “Red complex” pathogens (as noted above) have therefore been termed “Gateway” oral pathogens that create a systemic single dysbiotic disease state with multiple symptoms. The oral component houses the initial infection where the immune system is alerted and subverted, creating an inflammatory environment where circulating leukocytes carry these pathogens and associated virulence factors like lipopolysaccharides (LPS) and endotoxins such as gingipains via the circulatory system throughout the body. These affect the endothelial cells of arteries, and infect the arterial walls and neural tissues, compromising the blood brain barrier, heart tissue, the lungs, and the gut. The patient's health needs to therefore be thought of as a whole-body system with connections that originate in the oral cavity and if not properly and effectively controlled, have distant and highly damaging effects to multiple critical organ systems throughout the body.

Antibiotics have been used and continue to be used as antimicrobial agents to combat chronic oral periodontal pathogenesis. Systemically they are of limited use and even when applied topically in rinses or directly into the subgingival sulcus via for example, encapsulated microspheres, they are of limited efficacy and have historically had significantly serious and undesirable side effect profiles.

As an example, tetracyclines, including oral minocycline, have been associated with the development of autoimmune syndrome with symptoms such as joint pain, muscle pain, rash, swelling, fever, enlarged lymph nodes, and general body weakness and increased incidence of oral candidiasis (“thrush”). In clinical studies, the most frequently reported non-dental side effects were headache, infection, flu symptoms, and pain. The use of tetracycline class drugs, during tooth development may cause permanent discoloration of the teeth, and therefore should not be used in children or in pregnant or nursing women.

The antibiotic resistance of bacterial cells in biofilm has been reported to be 1,000 to 1,500 times greater than the resistance of planktonic (non-bound) bacterial cells in the oral cavity and has become a rising problem in recent years. Antibiotic resistance genes can be transferred between bacterial cells within biofilm resulting in a biofilm-wide resistance to the antibiotics. The widespread use of certain anti-microbials such as chlorhexidine to control these oral pathogens is problematic as chlorhexidine has equal and non-selective anti-microbial action against the many healthy commensal microbes in the oral cavity. Studies have linked its repeated use to alterations in taste sensation, parotid gland swelling, increased tartar formation on the teeth and hypertension. Oral biofilms treated with chlorhexidine exhibited a pattern of inactivation after only 24 hours with fast regrowth to the initial bacterial concentrations. Moreover, based on its non-selective anti-microbial action, chlorhexidine treatment induced profound shifts in microbiota composition and metabolic activity. In some cases, disease associated traits were increased (such as higher abundance of pathobiont strains or shift in high lactate production).

Polyphenols, a class of secondary metabolites abundant in Mediterranean foods, are pharmacologically active natural products with outstanding immunomodulatory actions. Upon binding to a range of receptors highly expressed in immune cells (e.g. AhR, RAR, RLR), they act in immuno-metabolic pathways through a mitochondria-centered multi-modal approach. First, polyphenols activate nutrient sensing via stress-response pathways, essential for immune responses. Second, they regulate mammalian target of rapamycin (mTOR)/AMP-activated protein kinase (AMPK) balance in immune cells and are well-tolerated caloric restriction mimetics. Third, polyphenols interfere with the assembly of NLR family pyrin domain containing 3 (NLRP3) in endoplasmic reticulum-mitochondria contact sites, inhibiting its activation while improving mitochondrial biogenesis and autophagosome lysosome fusion. Finally, polyphenols impact chromatin remodeling and coordinates both epigenetic and metabolic reprogramming.

Propolis is one of the natural substances made by bees for building and preservation of their hives. This resinous lipophilic material is sticky, soft, and flexible when exposed to heat but hard and breakable when cold. Propolis is primarily composed of resins (55-60%). Waxes and fatty acids contribute around 30-45% and aromatic oil and pollen about 5-10%. Other substances may include minerals, vitamins, polyphenols and flavonoids. The biological activity of propolis is mostly linked with flavonoids and hydroxycinnamic acid.

Research has revealed that it is difficult to standardize the chemical constituents and flavonoid contents of propolis as it is dependent on the environmental conditions at the site of collection, on its origin and type of plant pollen and species of bees that produced it. Depending on location, the chemical constituents of propolis include chrysin, galangin, pinocembrin, pinobaskin. These are flavonoids without B-ring substituents. The major component of temperate propolis is caffeic acid phenethyl ester. Similarly, the chemical composition of propolis originating from tropical regions includes prenylated phenylpropanoids (e.g., artepillin C), whereas propolis found in Pacific and African regions contains geranyl flavanones as the characteristic compounds.

It would therefore be advantageous in the present invention to incorporate in a preferred chewing gum formula or intra-oral muco-adhesive patch for treating the oral cavity itself, ethanolic extracts of propolis (EEP) that are reproducible (batch to batch and season to season) to overcome the inherent variability of propolis sources obtained from their natural state.

Ethanolic extract of propolis (EEP) does show high efficacy against the bacterial strains of bacteroides and peptostreptococcus. Propolis extracts also demonstrated excellent performance regarding in vitro tests against yeasts and propolis extracts demonstrated elevated levels of antiviral activity against herpes simplex virus-1 (HSV-1). Propolis extracts exhibited high anti-HSV-1 activity when the viruses were pre-treated with these drugs prior to infection. Anti-HIV-1 activity was observed with propolis samples from several geographic regions.

Propolis is reported to be a strong anti-inflammatory agent. In recent years, in vitro and in vivo studies have been performed on the Propolis effects on inflammation. Caffeic acid phenethyl ester (CAPE) is a major constituent of Propolis, which is derived from the honeybee hives and has been demonstrated to have significant anti-inflammatory, antibacterial, antiviral, antifungal, antioxidant, antioxidative and anticancer properties.

Research has proven that topical mouth rinses containing propolis in an alcohol aqueous solution heals intra-buccal surgical wounds; therefore, it plays a role in epithelial repair after tooth extraction and exerts an anti-inflammatory effect on orofacial pain. Propolis in toothpaste was seen to improve oral health and showed inhibitory effect on dental plaque formation, which is considered as the main etiology in the progression of most oral diseases.

In vitro studies have shown that propolis extracts (EEP) induced death of P. gingivalis cells by rapidly increasing membrane permeability of the bacterial cells and that antibacterial activity toward P. gingivalis was maintained even after extensive heat treatment, demonstrating a high level of thermostability, a useful characteristic for its use in the present invention.

Liposomal apigenin is active as an antioxidant, anti-inflammatory, anti-amyloidogenic, neuroprotective, and cognition-enhancing substance with interesting potential in the treatment/prevention of Alzheimer's disease. Apigenin possesses anti-obesity activity mainly by attenuating adipocyte differentiation by suppressing the mitotic clonal expansion and the adipogenesis-related factors, up-regulating the expression of multiple C/EBPβ inhibitors, and activating the COX2/PGE2 pathway for stimulation of UCP-1 via EP4 activation.

The sugarless sweetener and flavor enhancer Neohesperidin dihydrochalcone (NHDC) is a precursor for anthocyanins, contains polyphenols and has been reported to have various bioactivities, including antioxidant and hepatitis inhibitory effects. However, its anti-inflammatory functions and mechanisms of action are poorly understood. DHCA, a metabolite of NHDC significantly down regulated the secretion of pro-inflammatory cytokines. In contrast, NHDC had a marginal effect, suggesting that the biological metabolism of NHDC to DHCA is required for its anti-inflammatory function. However, both NHDC and DHCA rescued LPS-induced suppression of oxidative phosphorylation, which is a hallmark of anti-inflammatory M2 macrophages. 3T3-L1 adipocytes showed lower fat deposition in the presence of DHCA, while sugar-containing NHDC showed a slight increase in fat deposition. In high-fat diet-induced obese mice, treatment with NHDC successfully down-regulated body weight gain in a dose-dependent manner. Furthermore, M2 polarized bone-marrow-derived macrophages (BMDM) from NHDC-fed mice secreted an increased amount of the anti-inflammatory cytokine IL-10. Overall, these results indicate that NHDC and its physiological metabolite DHCA have the potential to suppress the inflammatory response and obese status. The inventors have observed that in composition for intra-oral administration comprising NHDC, upon release from chewing the gums it binds strongly to the oral tissues including the tongue and releases slowly over time (up to several hours). This provides a long-lasting sweet taste in the mouth so that even when drinking plain water, the water has a sweet taste.

The sugarless sweetener and taste modifier brazzein is derived from a berry. Brazzein is a sweet-tasting protein found in the fruit of the native West African Oubli plant (Pentadiplandra brazzeana). It is a soluble protein with a sweetness that is approximately 1500 times greater than sucrose. The protein is small in size, containing only 54 amino acids

Xylitol is a sugar alcohol that looks and tastes like sugar but has fewer calories and doesn't raise blood sugar levels. Xylitol consumption has been found to reduce S. mutans and S. sobrinus counts in saliva but appears not to reduce the numbers of S. sanguinis and S. mitis in saliva. So, habitual consumption of xylitol reduces cariogenic streptococci levels without any effect on beneficial streptococci for the oral cavity. All these may therefore also be incorporated as well into the compositions of the present invention. Xylitol may in particular be used for the final outer hard coating of the functional chewing gums.

Peppermint Oil was found to have anti-bacterial, antimicrobial, and antifungal properties. It is one of the most widely used essential oils because of its ability to inhibit oral biofilm and treat gum disease. Mentha, derived from Peppermint Oil is a potent tissue permeation enhancer as it is also a vasoactive agent that produces tissue vasodilation.

Green tea polyphenols, especially (−)-epigallocatechin gallate (EGCg) which is a dominant component of tea polyphenols, completely inhibited the growth and adherence of P. gingivalis onto the buccal epithelial cells at concentrations of 250-500 micrograms/ml. Among the polyphenolic compounds, (−)-epicatechin gallate (ECg) and (−)-gallocatechin gallate (GCg) were effective next to EGCg in these activities. On the other hand, (+)-catechin (C (+)), (−)-epicatechin (EC), (+)-gallocatechin (GC), and (−)-epigallocatechin (EGC) had very much less activity. These results indicate that the inhibitory effect on the adherence of P. gingivalis onto the buccal epithelial cells is attributed to the presence of the galloyl moiety, which is ester-linked with the 3-OH of the catechin moiety in the polyphenolic compounds.

Curcumin (CUM), also known as diferuloylmethane, is the main polyphenolic substance found in the rhizomes of Curcuma longa. CUM is recognized as having pleiotropic functions (anti-inflammatory, antioxidant, neuroprotective, immunomodulatory, antitoxic, anti-apoptotic, anti-diabetic (reduces insulin resistance), anti-fertility, antimicrobial, anti-allergic, anti-dermatophytic, antidepressant, and cardioprotective ones) and even when administered in large quantities, it has no major side effects. Curcumin inhibits the growth of periodontal pathogens (such as A. actinomycetemcomitans, F. nucleatum, and P. gingivalis) under planktonic and biofilm conditions.

Spirulina is a type of cyanobacteria-often referred to as blue-green algae, is a potent antioxidant with high polyphenol content. On usage, pro-inflammatory cytokines such as TNF-α, IL-1β, IL-6, and inflammatory transcription factor NF-κB were decreased in gingival tissue and osteogenesis-related factors (new bone growth around teeth) were promoted and BMP-2/Smad pathway was up-regulated in a periodontitis (gum disease) condition.

Coconut oil is a natural product from coconut containing polyphenols that has many benefits such as antibacterial, anti-inflammatory, and antioxidant The main etiology of periodontitis plaque of biofilm contains colonies of pathogenic microorganisms. The occurrence of inflammation in the periodontal tissue stimulates the release of inflammatory mediators, such as TNF-α and TGF-β. Treatment for periodontitis can be performed starting from initial therapy and usually accompanied by additional therapy such as local drug delivery.

L-theanine is an amino acid known for its calming effects. In the brain, 1-theanine increases dopamine, serotonin, and the inhibitory neurotransmitter glycine.

L-Carnitine helps move more fatty acids into your cells to be burned for energy, so it's sometimes used as a weight loss supplement. A 2020 review of 37 studies found that L-carnitine supplementation significantly reduced body weight, body mass index (BMI), and fat mass.

Conjugated Linoleic Acid (CLA) is a type of fat. Recent studies demonstrated that CLA supplementation reduces body weight, leptin and/or body adiposity in people.

Chrysin belongs to the group of natural polyphenols. It can be found, among others, in honey, propolis and fruits and has a wide range of biological activities, including the prevention of oxidative stress, inflammation, neurodegeneration and carcinogenesis. Chrysin plays an important role in prevention from cancer, oxidative stress, inflammatory disorders, diabetes mellitus, cardiovascular diseases, obesity, and allergic events.

Milk Thistle improves liver function and increases survival in people with cirrhosis or chronic hepatitis.

Black Seed Oil is exceptionally high in potassium, a mineral that diabetics are deficient in and that helps blood sugar patients with blood pressure control. It is also extraordinarily abundant in iron and the immunity-booster Vitamin C, both of which are critical for enhancing general health in diabetics.

Cayenne Pepper can also help relax the muscles in your blood vessels so blood can flow easily and act as a permeation enhancer for other materials to increase their bioavailability to the body.

L-Carnosine is able to counteract different factors, such as neuroinflammation, oxidative stress, and the deficit of neurotrophic factors which are strictly connected with aging-related cognitive decline and the risk to develop dementia. It exerts neuroprotective effects via modulation of the HO-1/Hsp72 system and by reducing neuronal damage caused by oxidative stress. It has biochemical properties, including antioxidant, bivalent metal ion chelating, muscular proton buffering, anti-cross-linking, and reactive carbonyl scavenger activities.

L-Histidine has been shown to protect against diseases related to brain aging such as brain infarction, cerebral ischemia, brain edema, sciatic nerve lesions, and neuropathic pain. It improves neurogenesis. In addition, protein expression levels of both neuronal markers (β tubulin-III and neurofilament heavy protein) and antioxidant enzymes, glutathione peroxidase-1 and superoxide dismutase-1 were up-regulated. Conversely, protein expression levels of amyloid β (1-42) and cleaved caspase-3 were down-regulated. Levels of mRNA for the pro-inflammatory cytokines, interleukin (IL)-8, IL-1β, and tumor necrosis factor-α were also down-regulated and may be used in the formulations of the present invention.

Astaxanthin rescued the number of surviving pyramidal neurons in the hippocampus. Lipid peroxidation (concentration of malondialdehyde) was decreased, and antioxidative capacity (levels of reduced glutathione and superoxide dismutase) in the hippocampus (seat of learning and memory in the brain) were increased.

Magnesium L Threonate slows down cognitive decline and brain aging by promoting synaptic plasticity and increasing the density of synapses in the hippocampus, a brain region critical for memory formation. It has been shown to provide a variety of benefits, including improved relaxation, increased focus, and better sleep quality. Is effective for cognitive and mental health conditions, like brain fog, mood disorders, migraines, and cognitive decline.

PQQ is pyrroloquinoline quinone. It is sometimes called methoxatin, pyrroloquinoline quinone disodium salt and is a powerful antioxidant. It is a compound made by bacteria and is found in fruits and vegetables. PQQ in bacteria helps them digest alcohol and sugar, which makes energy. This energy helps them survive and grow. Animals and plants don't use PQQ the same way that bacteria do, but it is a growth factor that helps plants and animals grow. It also seems to help them tolerate stress. PQQ supplements are often used for energy, memory, enhanced focus, and overall brain health and may be in the compositions of the present invention.

Research shows that phytochemicals present in cardamom seeds which is high in polyphenols can improve cognition, elevate mood, and protect brain cells from oxidative damage and inflammation. It can also reduce the accumulation of amyloid-beta plaques, which are a type of brain toxin associated with Alzheimer's disease and neurodegeneration and forms of it may be used in the formulations of the present invention.

Boron has been associated with proper brain function. Assessments of cognitive and psychomotor function in humans found that boron deprivation results in poorer performance on tasks of motor speed and dexterity, attention and short-term memory and may be included in compositions according to the present invention.

Gingko Biloba's root bark and leaves are rich in ginkgolide compounds, which help promote higher concentrations of acetylcholine in key regions of the brain responsible for decision-making and short-term memory recall. They are also beneficial for cerebral blood circulation and may be included in compositions according to the present invention.

Huperzine A has been shown to increase bioavailable levels of acetylcholine by inhibiting the enzyme that breaks it down, acetylcholinesterase. This fosters an ideal environment to form new neural connections and improve memory retention and may be included in compositions according to the present invention.

Pomegranate seed oil has proven helpful in improving cognitive function in multiple sclerosis patients experiencing cognitive difficulties associated with the disease. Consumption of anthocyanin-rich cherry juice for 12 weeks improves memory and cognition in older adults with mild-to-moderate dementia and may be included in compositions according to the present invention.

Gotu kola appears to support memory function, promote mental clarity, promote healthy stress levels and help support healthy cardiovascular function and may be included in compositions according to the present invention.

Aloe vera improves motor and memory performances as well as oxidative status of hippocampus and cerebral cortex. aloe has the unique advantage of being therapeutically effective in reducing oxidative damage, inflammation, increasing vasodilatation, treating tumors and neurodegenerative disorders, as well as an effective drug in maintaining general brain health and memory. Aloe has also been proven to possess cholinergic and cognitive enhancing capabilities. Today, aloe is used as an alternate medicine for diabetes, asthma, epilepsy, hepatic disease, HIV, cancer, osteoarthritis and may be included in compositions according to the present invention.

Vitamin D is an important calcium-regulating hormone with diverse functions in numerous tissues, including the brain. Increasing evidence suggests that vitamin D may play a role in maintaining cognitive function and that vitamin D deficiency may accelerate age-related cognitive decline and may be included in compositions according to the present invention.

Niacinamide protects brain cells from stress and injury. The deficiency of this vitamin causes a decline in brain function, manifested as memory loss and dementia. Niacinamide intake is likely to be beneficial for brain health. Niacin promotes the growth and development of brain cells (neurons). In fact, brain fog and even psychiatric symptoms are associated with niacinamide deficiency and may be included in compositions according to the present invention.

Vitamin B12 deficiency has been associated with memory loss, especially in older adults. One study in people with early-stage dementia showed that vitamin B12 may help slow cognitive decline. It is important for producing serotonin and dopamine, which are mood-enhancing neurotransmitters. As a result, vitamin B12 can help to prevent and treat depression and anxiety. It can improve focus and memory as well and may be included in compositions according to the present invention.

Folic Acid (Folate) aids in the creation of DNA and RNA, formation of neurotransmitters, and the formation of the nervous system during pregnancy. Folate is also known to help with depression, mental fatigue, and irritability because it can be quickly broken down and supply the body with energy and may be included in compositions according to the present invention.

Zinc promotes antioxidant effects, neurogenesis, and immune system responses. From neonatal brain development to the preservation and control of adult brain function, zinc is a vital homeostatic component of the CNS. It is highly concentrated in the amygdale, the auditory brain stem, the cerebral cortex, and the hippocampus. Zn functions in the brain as a neurotransmitter and second messenger, controlling hippocampus long-term potentiation, boosting neuronal survival, and promoting learning and memory and may be included in compositions according to the present invention.

Alpha lipoic acid (ALA), a powerful antioxidant, has the potential to relieve age-related cognitive impairment and neurodegenerative disease. Clinical randomized controlled studies have demonstrated the cognitive improvement effects of lipoic acid in Alzheimer's disease and may be included in compositions according to the present invention.

Alpha GPC naturally occurs in the body as a precursor to acetylcholine, one of the key neurotransmitters in the brain's neural network. As a supplement, this compound rapidly crosses the blood-brain barrier and helps promote the synthesis of acetylcholine. In addition, alpha GPC encourages the development of cell membranes in the cerebral cortex, or “gray matter,” for improved mental processing and may be included in compositions according to the present invention.

Bacopa monnieri is an herb commonly used in traditional Ayurvedic medicine for its positive influence on memory and focus. Studies indicate the bacoside compounds contained in this potent plant cross the blood-brain barrier and, like huperzine A, help boost levels of acetylcholine by limiting its breakdown by acetylcholinesterase. In this way, bacopa monnieri helps generate new neural pathways and may be included in compositions according to the present invention.

Marigold is rich in both lutein and zeaxanthin. These may help to boost memory and cognitive function. Marigold extract also has antioxidant and anti-inflammatory properties, which should protect the brain against stress and aging and may be included in compositions according to the present invention.

The amino acid Tryptophan is important for cognitive processes because of its role in serotonin production. Low levels of this amino acid can impair cognition, including memory of events or experiences and may be included in compositions according to the present invention. L-tryptophan is an essential amino acid that helps the body make proteins and certain brain-signaling chemicals. Your body changes L-tryptophan into a brain chemical called serotonin. Serotonin helps control your mood and sleep.

L-citrulline and L-arginine which promote the production of Nitric Oxide.

Quercetin is a plant-derived flavonoid, and has shown neuroprotective effects against neuro-inflammation. Improvement against Aβ25-35-induced memory loss and cognitive decline. In another study, it attenuated cell death (apoptosis) caused by hydrogen peroxide in neuronal cell lines. It reshapes gut microbiota homeostasis and modulates brain metabolic profile by free radicals scavenging and enhancing the antioxidant mechanisms in the brain mitochondria. It reversed memory impairment via attenuating IL-6 and TNF-α brain levels in the brain and provides protection against damages to the hippocampal brain regions and prefrontal cortex. It reversed neurodegeneration in the hippocampal brain regions and prefrontal cortex and mitigates pro-inflammatory mediators and reverses neurodegeneration to restore memory function.

Coenzyme Q10: Attention and executive function impairment were significantly explained by the increase of oxidative stress accompanied by a decrease in CoQ10 levels. Other studies indicate that CoQ10 supplementation improves cognitive function and induces neuroprotective effects and enhances cellular mitochondrial ATP production. Adding bioperidine (Black Pepperdine) as a permeation enhancer increases the absorption of CoQ10 and may be used to enhance the permeation of other ingredients thereby increasing their absorption and bioavailability to the body.

Benfotiamine has shown beneficial effects in treatment of various disorders, most notably thiamine deficiency, diabetes, alcoholism and neurodegenerative diseases including Alzheimer's disease. These effects have been investigated in a plethora of in vitro and in vivo models.

The intra-oral composition of the present invention may incorporate ingredients/agents/drugs that can be utilized to treat obesity, stress, insulin resistance/type II diabetes, cardiovascular disease, chronic kidney disease, rheumatoid arthritis, metabolic syndrome, gut microbiome dysbiosis, Crohn's disease.

Some examples of substances that may be used in the formulations of the present invention to treat or prevent obesity, insulin resistance/type II diabetes, gut metabolic dysbiosis, metabolic syndrome, and Crohn's disease include (but are not limited to): a. GLP-1 receptor agonists (e.g. semaglutide). The mechanisms through which semaglutide delivers its benefits in lowering blood glucose levels and promoting weight loss involve the activation of GLP-1 receptors primarily located in the gastrointestinal tract, pancreas, and brain. By decreasing the production of glucagon, which elevates blood glucose levels, semaglutide aids in lowering the body's sugar output. This reduction in glucagon not only helps in managing blood sugar but also supports weight management by decreasing the body's need to store excess glucose as fat. b. As one of the important metabolites of purines, the function of inosine and its transmembrane transporter, ENT1, in promoting the thermogenic program and energy expenditure (EE) of brown adipose tissue (BAT) bring a new hope to weight loss in BAT-centered obesity therapies due to the latest findings. c. Berberine improves physiological stimulation of glucose via cascade reaction of insulin-like growth factor-1 (IGF-1), thus inducing secretion of insulin in the body, reducing insulin resistance, and improving sensitivity of liver, muscle tissues and fat to insulin. d. Naringin supplementation improved glucose intolerance and insulin resistance in a model of high-fat-diet-fed mice and increased glucose uptake by skeletal muscle cells in an AMPK-dependent manner. Naringin has anti-inflammatory and antioxidant benefits in diabetic nephropathic rats, as evidenced by the downregulation of IL-1, proinflammatory cytokines TNF, and IL-6 and the upregulation of antioxidants SOD, GSH, and CAT. It is also very effective for weight loss where supplement intake of naringin reduced caloric intake by ˜14% and total adiposity decreased by approximately 50%. Additionally, naringin significantly reduced perigonadal adipose tissue mass, even after controlling for body weight. e. Rosmarinic acid is a powerful polyphenol that has been found to act like insulin to lower overall glucose levels. f. Green coffee extract may act by lowering blood sugar and blocking fat buildup. Green coffee also seems to help lower high blood pressure in some people. g. Fenugreek may increase insulin sensitivity by enhancing insulin action at the cellular level, lowering HbA1c levels by using glucose in the peripheral tissues and maintaining blood glucose levels. A study with group taking fenugreek as a dietary supplement found a significant reduction in fasting plasma glucose (FPG), postprandial plasma glucose (PPPG) and low-density lipoprotein cholesterol (LDL cholesterol) whereas serum insulin increased significantly. h. The use of Ephedra promotes weight loss in selected populations. In healthy overweight and obese populations Ephedra decreased body weight, fasting glucose levels and insulin levels. These findings indicate that Ephedra decreases the risks of glucose intolerance and obesity. i. 7-keto DHEA demonstrated effects in improving metabolic disorders related to weight and insulin resistance. One study, for example, evaluated if 7-keto DHEA reduced abdominal fat and improved insulin activity in older adults. Results indicated that 7-keto DHEA replacement decreased fat and lowered insulin levels. j. Vitamin C does not directly lead to fat oxidation or loss of body fat. However, it is related to body weight and waist circumference. One study found that vitamin C and body mass are inversely related, meaning low plasma ascorbic acid concentrations are linked to high body mass index (BMI). Among the possible beneficial effects of ascorbic acid on obesity-related mechanisms, it has been suggested that this vitamin may: (1) modulate adipocyte lipolysis; (2) regulate the glucocorticoid release from adrenal glands; (3) inhibit glucose metabolism and leptin secretion on isolated adipocytes; (4) lead to an improvement in hyperglycemia and decrease glycosylation in obese-diabetic models; and (5) reduce the inflammatory response. Possibly, all these features could be related with the outstanding antioxidant characteristics of this vitamin. k. alpha lipoic acid.

According to an embodiment, the topical application time of the intra-oral composition should preferably be in the range of 5-20 minutes of Total Exposure Time (TET). Compositions for intra-oral delivery according to embodiments, diffuse and penetrate the oral tissues in this relatively brief period of time and provide to the subject relatively large amounts of active agent. Such administration is of immense clinical value and health benefit to the user as user compliance can be problematic for many and is a major factor to consider and overcome.

The following examples are provided to illustrate certain particular features and/or embodiments. These examples should not be construed to limit the disclosure to the features or embodiments described.

### EXAMPLES

### Example 1: Hydration of EM with and without PCM

An experiment was performed to determine heat of hydration of anhydrous salts with and without PCM.

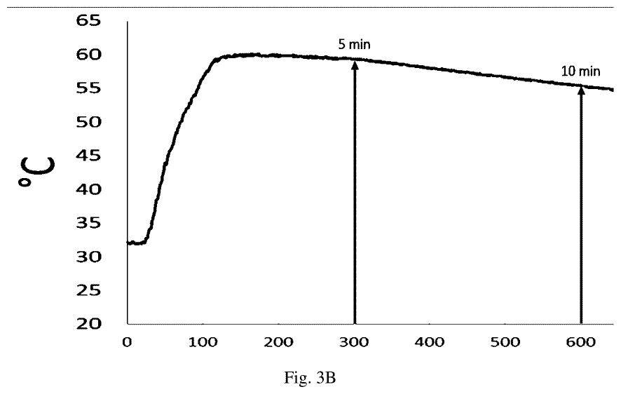

For testing anhydrous salts without PCM, 18 grams (g) of anhydrous magnesium citrate was combined with 8 g of anhydrous magnesium sulfate (26 g total), with 74 g distilled water. For testing anhydrous salts with PCM, the same concentrated blend of anhydrous salts was added to the same amount of water, along with 2.6 g of PCM in the form of a 1:1 weight ratio blend paraffin oil:beeswax. The ratio of PCM to anhydrous salt was 1:10 ratio. Temperature of the mixtures was measured over time and can be shown in FIGS. 3A (no PCM) and 3B (with PCM).

As can be seen in FIG. 3A, the anhydrous blend of EMs alone (without PCM) rapidly reaches peak maximum temperature in about 2 minutes and cools rapidly by 5° C. in the first 5 minutes and then cools a total of 12° C., over the first 10 minutes. By comparison in FIG. 3B, the same fully anhydrous blends of EMs with the additional PCM rapidly reaches peak maximum temperature and cools gradually by 2° C., over the first 5 minutes and then cools a total of only 5° C., over the first 10 minutes. This particular combination of the EMs with the PCMs improves the heat retention of the solution by a factor of 2.5 during the first five minutes of the hydration reaction and an improves heat retention factor of 3.0 during the first ten minutes of the hydration reaction. This data indicates that compositions for intra-oral delivery comprising cores with EM and PCM will be able to maintain heat in the oral period for longer periods than equivalent compositions having EM without PCM.

### Example 2: Chewing Gum Composition Comprising Active Agents, PCM and EM

Chewing gum formulations for delivering active agents via intra-oral delivery were prepared. The amount of gum base used was 34%, and the gum had 66% other ingredients.

The gum base was a low-ash content gum base, having ash content of 5%+/−2 by weight. The gum base comprises synthetic polymers/elastomers, plasticizers, and resins.

Cores prepared from the ingredients can be coated in a water free pre-coat, using one of or a blend of any of the following: either coating the center core with sugar free shellac and then precoating for another coating with a sugar free gumming agent that smooths the center core surface and allows for homogeneous coating results, even for cores with uneven surfaces. Alternatively, coat with shellac, or surface treatment with a sugar free liquid glazing and sealing agent and pre-coating with sugar free shellac for a further coating. The overall ratio, by weight of core to coatings was 8:2 or 7:3

These core compositions provided heat to the oral cavity while being chewed by a subject and kept chewing gum elasticity throughout the chewing for at least ten minutes.

Described herein, according to an embodiment, are compositions for intra-oral delivery of active agents comprising: a core, the core having at least one active agent, a phase change material (PCM), and an exothermic material, the core comprising less than 0.5% water.

Optionally, the exothermic material is in the form of an anhydrous pharmaceutically acceptable or food grade salt. Optionally, the exothermic material is in the form of an anhydrous salt selected from the group consisting of: magnesium citrate, magnesium sulfate, strontium bromide (SrBr₂), strontium chloride (SrCl₂), trisodium phosphate (Na₃PO₄), magnesium chloride (MgCl₂), calcium sulfate (CaSO₄), and zinc sulfate (ZnSO₄). Optionally, the exothermic material is in the form of an anhydrous salt selected from the group consisting of: magnesium citrate and magnesium sulfate. Optionally, the composition core comprises 10% to 25% of exothermic material. Optionally, the active agent is selected from the group consisting of: a drug and a nutritional supplement. Optionally, the active agent is selected from the group consisting of: glucagon like peptide-1 (GLP-1) receptor agonists, beta blockers, calcium channel blockers, ACE inhibitors, opioids, NMDA receptor antagonists, hormones, phosphodiesterase 5 (PDE 5) inhibitors, opioid antagonists, H1 receptor antagonists, and benzodiazepines. Optionally, the GLP-1 receptor agonist may be selected form the group consisting of: semaglutide, dulaglutide, exenatide, liraglutide, tirzepatide, and lixisenatide. Optionally, the opioid may be selected from the group consisting of: morphine, loperamide and oxytocin. Optionally, the MDA receptor antagonist may include ketamine. Optionally, the hormone may be selected from the group consisting of: progesterone and testosterone. Optionally, the PDE 5 inhibitor may be selected from the group consisting of: sildenafil and tadalafil. Optionally, the opioid antagonists may include naltrexone. Optionally, the H1 receptor antagonist may include promethazine. Optionally, the opioid receptor agonists may include loperamide. Optionally, the benzodiazepine may include triazolam. Optionally, the composition core comprises above 15% active agent by weight. Optionally, the composition core comprises between 20% and 40% active agent by weight. Optionally, the PCM undergoes phase change from solid to liquid at a temperature between 40° C. to 48° C., optionally, between 44° C. to 48° C. Optionally, the PCM is present in the composition core in an amount of between 1% and 10% by weight. Optionally, the PCM is present in the composition core in an amount of 1.5-2% by weight. Optionally, the PCM is selected from the group consisting of: beeswax, glycerol, polyethylene glycol, and paraffin oil. Optionally, the PCM comprises Beeswax and paraffin oil. Optionally, the beeswax and paraffin oil are present in a ratio of between 1:1 to 2:3. Optionally, the composition further comprises a permeation enhancer. Optionally, the permeation enhancer is selected from the group consisting of: mannitol, menthol, bioperidine, cayenne pepper and an essential oil. Optionally, the essential oil is selected from the group consisting of: peppermint oil, sage oil, and eucalyptus oil. Optionally, the permeation enhancer is present in the composition core in an amount of between 1% and 5% by weight. Optionally, the composition further comprises an ingredient selected from the group consisting of: a sweetener, a bitterness blocker, and a flavoring. Optionally, any one or more than one of the PCM, exothermic material, and active agent are microencapsulated. Optionally, the composition comprises at least one coating layer surrounding the core. Optionally, the coating comprises a pre-coating layer, which serves as a moisture barrier. Optionally, the pre-coating layer comprises beeswax, carnauba wax, candelilla wax, sumac wax, sunflower wax or cocoa butter. Optionally, the composition further comprising a final coat layer. Optionally, the weight ratio of core to composition is between 8:2 and 7:3. Optionally, the composition is in the form of a gum, and further comprises a gum base. Optionally, the gum base comprises 5%+/−2 by weight ash content. Optionally, the salt content of the composition is no more than 30% by weight. Optionally, the composition is in the form of a muco-adhesive patch. Optionally, the composition further comprises a muco-adhesive layer and a semi-permeable membrane layer surrounding the core.

Further described herein, according to an embodiment, is a method for administering an active agent to a subject, comprising introducing an active agent using a composition comprising: a core, the core having at least one active agent, a phase change material (PCM), and an exothermic material, the core comprising less than 0.5% water; into the oral cavity of the subject, and contacting the composition with saliva, thereby generating heat within the oral cavity of the subject.

Further described herein, according to an embodiment, is a method for treatment or prevention of a disease or condition or for optimization of health; comprising administering to the subject via the intra-oral route, active agents using a composition comprising: a core, the core having at least one active agent, a phase change material (PCM), and an exothermic material, the core comprising less than 0.5% water; to a subject in need thereof. Optionally, the disease or condition is selected from a group consisting of: periodontal disease (periodontitis, peri-implantitis), recurrent aphthous ulcers, oral candidiasis, caries, halitosis, cardiovascular disease, respiratory disease, Diabetes mellitus, Alzheimer's Disease, Fronto-Temporal dementia, dementia, Parkinson's Disease, microbial disease, biofilm formation, inflammation, neuroinflammation, cognitive decline, brain infarction, cerebral ischemia, brain edema, sciatic nerve lesions, neuropathic pain, mood disorders, improved mental performance/mental acuity, memory recall, obesity, stress, insulin resistance/type II diabetes, chronic kidney disease, rheumatoid arthritis, metabolic syndrome, gut microbiome dysbiosis, and Crohn's disease.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An intra-oral composition for intra-oral delivery of active agents comprising: a core, the core having at least one active agent, a phase change material (PCM), and an exothermic material, the core comprising less than 0.5% water.

2. The composition according to claim 1 wherein the exothermic material is in the form of an anhydrous pharmaceutically acceptable or food grade salt.

3. The composition according to claim 2 wherein the exothermic material is in the form of an anhydrous salt selected from the group consisting of: magnesium citrate, magnesium sulfate, strontium bromide (SrBr₂), strontium chloride (SrCl₂), trisodium phosphate (Na₃PO₄), magnesium chloride (MgCl₂), calcium sulfate (CaSO₄), and zinc sulfate (ZnSO₄).

4. The composition according to claim 3 wherein the exothermic material is in the form of an anhydrous salt selected from the group consisting of: magnesium citrate and magnesium sulfate.

5. The composition according to claim 1 wherein the composition core comprises 10% to 27% of exothermic material.

6. The composition according to claim 1, wherein the active agent is selected from the group consisting of: a drug and a nutritional supplement.

7. The composition according to claim 1 wherein the active agent is a drug selected from the group consisting of: glucagon like peptide-1 (GLP-1) receptor agonists, beta blockers, calcium channel blockers, ACE inhibitors, opioids, NMDA receptor antagonists, hormones, phosphodiesterase 5 (PDE 5) inhibitors, opioid antagonists, H1 receptor antagonists, and benzodiazepines.

8. The composition according to claim 7 wherein the drug is a GLP-1 receptor agonist selected form the group consisting of: semaglutide, dulaglutide, exenatide, liraglutide, tirzepatide, and lixisenatide.

9. The composition according to claim 1 wherein the composition core comprises above 15% active agent by weight.

10. The composition according to claim 1 wherein the PCM undergoes phase change from solid to liquid at a temperature between 40° C. to 48° C.

11. The composition according to claim 1 wherein the PCM is present in the composition core in an amount of between 1% and 10% by weight.

12. The composition according to claim 1 wherein the PCM is selected from the group consisting of: beeswax, glycerol, polyethylene glycol, and paraffin oil.

13. The composition according to claim 12 wherein the PCM is a combination of beeswax and paraffin oil and are present in a ratio of between 1:1 to 2:3.

14. The composition according to claim 1, further comprising a permeation enhancer.

15. The composition according to claim 14 wherein the permeation enhancer is selected from the group consisting of: mannitol, menthol, black pepperdine, cayenne pepper and an essential oil.

16. The composition according to claim 1, further comprising an ingredient selected from the group consisting of: a sweetener, a bitterness blocker, and a flavoring.

17. The composition according to claim 1 further comprising at least one coating layer surrounding the core, comprising a pre-coating layer, which serves as a moisture barrier.

18. The composition according to claim 17, further comprising a final coat layer.

19. The composition according to claim 1, in the form of a gum, and further comprising a gum base.

20. The composition according to claim 1 wherein the salt content of the composition is no more than 30% by weight.
